(19) United States
(12) Patent Application Publication
(10) Pub. No.: US 2023/0381469 A1
(43) Pub. Date: Nov. 30, 2023

(54) DRUG DELIVERY BALLOON CATHETER

(71) Applicant: THE CHLOROPLASTPTE. LTD., Singapore (SG)

(72) Inventors: Jingnan Luo, Singapore (SG); Bo Huai Moses Lee, Singapore (SG)

(21) Appl. No.: 18/033,528

(22) PCT Filed: Nov. 16, 2021
(86) PCT No.: PCT/SG2021/050708

§ 371 Date: Apr. 24, 2023

Related U.S. Application Data
(60) Provisional application No. 63/114,771, filed on Nov. 17, 2020.

(51) Int. Cl. A61M 25/10; A61M 25/00

(52) U.S. Cl. CPC A61M 25/1018; A61M 25/1025; A61M 25/0084

(57) ABSTRACT

Provided are drug infusion catheters comprising an axially extending elongate member, an inflatable balloon, two or more cannula each housing an extendable needle. The drug infusion catheters are useful in the delivery of drugs to tissue surrounding a lumen or vessel within a body.

### FIELD OF THE INVENTION

The present invention relates to a device, i.e. a catheter (e.g. a rapid exchange catheter or an over the wire catheter), for the delivery of drugs to the body, particularly to the tissue surrounding a physiological lumen or vessel.

### BACKGROUND OF THE INVENTION

Many methods can be used to deliver drugs into the body. Examples include oral, submucosal, parenteral, and transdermal administration. However, these methods generally suffer from non-specific delivery, which results in a potential for off-target side effects. Since drug delivery efficiency is crucial in disease treatment, focused drug delivery could provide improved outcomes with fewer side effects.

Technology that provides drug delivery almost exclusively to the desired site can be referred to as smart drug delivery. Smart drug delivery improves the bioavailability of the drug at the site of disease, thus, reducing dosing frequency, and minimizing side effects resulting from systemic administration. There are many possible methods to achieve smart drug delivery, for example the use of nanoparticle carriers which have strong affinity to specific proteins and can control where the therapeutic agent ends up. However, a drawback with this approach is that the target site needs to be distinct enough for the nanoparticle carriers to differentiate between the target site and other locations. This means that such an approach cannot be used for every case.

Injecting the therapeutic agent directly into the region to be treated is one of the simplest methods to control delivery of a therapeutic agent. However, this method is less simple when the target site is not easily accessible. For injection a needle is generally inserted from outside the body, penetrating through the skin and other tissue, until the needle tip reaches the target position. The needle is then used to inject the therapeutic agent directly into the target. The main limiting factor in this approach is whether the needle can reach the target site. Some areas, such as areas covered by bone, cannot be reached using this method. Another drawback of this approach is that the needle has to pass through much of the patent's tissues in order to reach the target site, so it can be difficult to achieve very high levels of precision.

When a target is deep within the body tissue, the approach can be improved by navigating a needle apparatus through the vascular network to reach the target vessel before deploying needles to penetrate the wall of the target vessel to reach the target site. The walls of blood vessels are a prime target for smart drug delivery due to the proximity of blood vessels to almost every area in the body. Injecting therapeutic agents directly into vessel walls in a circumferential manner could allow effective treatment of a number of disorders, such as vascular diseases, arterial restenosis and also the promotion of angiogenesis in the ischemic heart. This delivery method could also be used with neuroablative agents to achieve nerve ablation. Examples of diseases which can be treated by ablating the nerves surrounding blood vessels are hypertension and diabetes, in which the target vessels for denervation are the renal and hepatic artery respectively.

Certain devices for vascular injection are known in the art and are discussed below.

The Bullfrog® micro infusion catheter described by Seward et al in U.S. Pat. Nos. 6,547,803 and 7,666,163, consists of a single needle attached to an inflatable elastic balloon. When the inflatable elastic balloon is inflated within the vasculature, the needle is forced against the wall of the blood vessel, thus penetrating the inner wall. The needle can then be used for the injection of a therapeutic or neuroablative agent. Seward et al also describes another embodiment with two needles, but such a system would be difficult to miniaturize and smaller vessels cannot be accessed. Although the Bullfrog® micro infusion catheter is able to deliver medical or neuroablative agent into the target vessel, there are several drawbacks when it is used to treat vascular diseases and for ablation. The first drawback is that if only one needle is used, it would require multiple applications to achieve circumferential delivery in the target vessel. Even then, controlled and accurate rotation of the distal end of any device via manipulation at the proximal end is difficult at best and would likely lead to irregularly spaced injections. The second drawback is that The Bullfrog® micro infusion catheter does not allow for precise, controlled and adjustable penetration depth depends on the balloon inflation. The third drawback is the limit to the maximum depth penetration achievable. The maximum depth penetration depends on the needle length but the maximum length of the needle is limited since it will affect the profile of the device. The inability to inject agents to an adequate depth limits the available treatment the device can provide.

Chow et al in U.S. Pat. No. 6,692,466 and Chan et al n U.S. Pat. No. 7,273,469 disclose a catheter with retractable needles housed in guide tubes, for drug injection into tissue. The distal portion of the guide tube is affixed to a portion of an expandable member and while the proximal portion of the guide tube is affixed to the tubular member which the expandable member is coupled to. Although the device described would be able to achieve circumferential delivery at a precise, controlled and adjustable depth, several drawbacks are also present. The first drawback is that affixing the guide tube to the balloon and tubular member limits the region the guide tube bends as the guide tube has to follow the profile of the balloon. This abrupt change in the direction of the guide tube results in a small bending radius leading to an increased force required to displace the needle past the curvature. The increased force increases the likelihood of the needle being damaged or puncturing the guide tube. The second drawback is that the material used for the guide tube is limited. Due to the small bend radius of the guide tube, the material used for the guide tube must be flexible. Chan et of mentioned the use of a sheath ring to prevent delamination of the guide tube, however, even the use of such a structure would still require the guide tube to be flexible. This requirement for the guide tube limits the material to polymers and prevents the use of metals and alloys. The third drawback is that the device would have difficulty achieving a high penetration depth to needle advancement ratio. The optimal penetration depth to needle advancement ratio is 1:1, and this is only achievable if the needle is perpendicular to the axis of the catheter. In the device described, this ratio is governed by the balloon cone angle which is typically between 20 and 40 degrees. An approach to improve the ratio without changing the balloon cone angle would be through the use of needles which are curved at the distal end. However, since Chow and Chan cannot use hard materials for the guide tube, they cannot use curved needles. Further, Chow and Chan describe the use of ribbons/deflectors made of a hard material to prevent the puncture of the guide tube at the bend, but a curved needle would have the needle point against the guide tube along the entire length of movement making it impractical to use ribbons/deflectors on curved needles.

The Peregrine System™ described by Fischell et al in U.S. Pat. No. 8,740,849 uses three equally spaced needle guiding elements which are moved outwardly to center the catheter before deploying needles from within each of the needle guiding elements. Neuroablative agent agents can then be delivered through the needles to achieve circumferential ablation of the target vessel. Although the Peregrine System™ is be able to achieve circumferential delivery at a precise, controlled and adjustable depth, there are several drawbacks regarding the use of needle guiding elements for the centering of the catheter within the target vessel. During the centering process of the Peregrine System™ within the target vessel, the needle guiding elements are extended simultaneously at the same rate until all the needle guiding elements all come into contact with the inner wall of the target vessel. This method of centering works well for rounded vessel such as arteries but for vessels which have an irregular or flattened cross sectional shape such as veins, this method for centering would not achieve the target result of a circumferential deposition of medical agents within the vessel. If the needle guiding elements are used to force the target vessel to adopt a circular shape, there is the risk of severe vessel damage due to the small area of contact provided by the needle guiding elements. The second drawback is risk of damage to the vessel wall resulting from the high pressure that may result between the point of contact of the hollow needle guiding elements and the vessel wall. The third drawback is the risk of catheter repositioning after the needle guide elements have been deployed. Unless the pressure between the needle guiding elements and the vessel wall is high, the few points of contact between the needle guiding elements and the vessel wall is likely unable to provide sufficient catheter securement. If the proximal end of the catheter is accidently moved, it is possible for the distal end of the catheter to be shifted. If this occurred before needle deployment, only a simple repositioning would be required. However, severe damage to the vessel can result if such an event occurred when the needles are deployed. The fourth drawback is the difficulty to achieve the same penetration depth for all needles. The points of contact provided by needle guiding elements all lie within a single plane. Since both the needle guiding elements and needles of the Peregrine System™ have a preset curved shape, the plane of contact has to be perpendicular to the target vessel in order for all the needles to achieve the same penetration depth within the target vessel. If a procedure requires the medical agents to be delivered to a precise depth, it would be challenging to use the Peregrine System™ to achieve the required precision for all the injection points.

There is a need for an injection catheter ha overcomes the problems of the prior art discussed above.

### SUMMARY OF THE INVENTION

The present invention provides a catheter which can be used to deliver substances into tissue outside of physiological lumens. The catheter may be used to deliver therapeutic agents to prevent and/or treat a number of diseases, such as vascular diseases, arterial restenosis, to ischemic heart disease, and diseases requiring nerve ablation, such as hypertension, diabetes or chronic obstructive pulmonary disease.

The present invention therefore provides the following.

1. A drug delivery device comprising:
an axially extending elongate member having a proximal portion, a distal portion, a first lumen and a second lumen;
an inflatable balloon coupled to the distal portion of the axially extending elongate member, the inflatable balloon having a proximal end and a distal end that are coupled together by a working portion, the balloon also having an exterior surface and an interior surface that defines a balloon chamber, where the first lumen of the axially extending elongate member is in fluid communication with the balloon chamber;
two or more guiding cannulas, each having a proximal portion and a distal portion, where the proximal portion of each guiding cannula is coupled to the axially extending elongate member,
two or more needles, each having a lumen and being housed within one of the two or more guiding cannulas, such that each needle is reversibly extendable from the distal portion of the guiding cannula that houses it, and where the second lumen of the axially extending elongate member is fluidly connectable to the lumen of each needle, wherein,
when the inflatable balloon is inflated, the distal portion of each of the two or more guiding cannulas do not extend beyond an outermost diameter of the exterior surface of the inflatable balloon.

2. The device of Clause 1, wherein the inflatable balloon is coupled to the distal end of the axially extending elongate member.

3. The device of Clause 2, further comprising a balloon support element within the inflatable balloon, optionally wherein the balloon support element is coupled to the distal portion of the axially extending elongate member and to the distal end of the inflatable balloon.

4. The device of Clause 1 or 2, wherein the axially extending elongate member comprises a third lumen, optionally wherein the third lumen is suitable for mounting the device on a guide wire.

5. The device of Clause 1 or 4, wherein the inflatable balloon is disposed over the distal portion of the axially extending elongate member.

6. The device of any one of the preceding clauses, wherein the distal portion of each guiding cannula is able to move freely with respect to the inflatable balloon, and/or wherein the distal portion of each guiding cannula is not affixed to the inflatable balloon.

7 The device of any one of the preceding clauses, wherein the guiding cannula are disposed around the circumference of the axially extending elongate member at substantially evenly spaced intervals.

8. The device of any one of the preceding clauses, wherein the distal portions of the guiding cannulas are configured to contact the exterior surface of the inflatable balloon and move with the exterior surface of the inflatable balloon as the inflatable balloon is inflated.

9. The device of Clause 8, wherein the guiding cannulas are configured to at least partially revert back to their original shape when the inflatable balloon is deflated.

The device of any one of the preceding clauses, wherein the inflatable balloon has a proximal cone portion, a central cylindrical portion and a distal cone portion.

11. The device of Clause 10 as dependent upon Clause 8, wherein the distal portions of the guiding cannulas are configured to contact the exterior surface of the proximal cone portion of the inflatable balloon.

12. The device of any one of the preceding clauses, wherein each guiding cannula comprises slits cut into one side of the guiding cannula.

13. The device of any one of the preceding clauses, wherein the two or more guiding cannulas are made from a metal or alloy.

14. The device of any one of the preceding clauses, further comprising a sheath movably disposed over the axially extending elongate member.

15. The device of Clause 14, wherein the sheath has an extended state and a retracted state, and wherein in the extended state the sheath covers the entirety of the two or more cannula and at least part of the inflatable balloon, and in the retracted state the sheath does not cover a part of the inflatable balloon, optionally wherein in the retracted state the sheath covers only part of the two or more cannulas.

16. The device of any one of the preceding clauses, wherein the needles are simultaneously reversibly extendable.

17. The device of any one of Clauses 1-15, wherein the needles are independently reversibly extendable.

18. The device of Clause 1, wherein each needle comprises a curved end portion, optionally wherein each needle is configured such that when the needle is extended from the cannula that houses it, the curved end portion curves outwardly in the radial direction from the axially extending elongate member.

19. The device of any one of the preceding clauses, wherein the second lumen of the axially extending elongate member comprises two or more sub-lumens, each sub-lumen being fluidly connectable to the lumen of one of the two or more needles, optionally wherein the two or more sub-lumens are not in fluid communication with each other.

20. The device of any one of the preceding clauses, further comprising a support structure for positioning and securing the guiding cannulas to the axially extending elongate member.

21. The device of any one of the preceding clauses, wherein the distal end of the axially extending elongate member is coupled to a flexible tip.

22. The device of any one of the preceding clauses, wherein the device is a rapid exchange or over the wire catheter.

23. The device of any one of the preceding clauses, comprising at least one radiopaque element, optionally wherein the radiopaque element is positioned within the balloon chamber, on the axially extending elongate member, on a guiding cannula, and/or on a needle.

24. The device of any one of the preceding clauses, further comprising a handle disposed at the proximal end of the axially extending elongate member.

The device of Clause 24, wherein the handle comprises a mechanism for controlling the maximum needle extension depth.

26. The device of Clause 24 or 25, wherein the handle comprises a means for reversibly extending the needles from the cannulas.

27. The device of Clause 26, wherein the means for reversibly extending the needles from the cannulas comprises a first means for roughly reversibly extending the needles and a second means for finely reversibly extending the needles.

28. The device of any one of Clauses 24 to 27, wherein the handle comprises a visual indicator of needle extension depth.

29. The device of any one of the preceding clauses, wherein the two or more guiding cannulas comprise a curved portion at their distal ends, optionally wherein the two or more guiding cannulas each comprise an opening facing outwards from the catheter at their distal ends.

30. The device of any one of the preceding clauses, wherein:
each needle comprises a curved end portion;
the distal portions of the guiding cannulas are configured to contact the exterior surface of the inflatable balloon and move with the balloon exterior as the balloon is inflated; and
the guiding cannulas are able to at least partially revert back to their original shape after the balloon is deflated,
optionally wherein each needle is configured such that when the needle is extended from the cannula that houses it the curved end portion curves outwardly in the radial direction from the axially extending elongate member.

The current invention has several benefits over the devices described prior. By using retractable needles, the device is able to achieve a greater range of penetration depths in a controlled manner. When compared to the Peregrine System™ described by Fischell et al in U.S. Pat. No. 8,740,849, the use of a balloon instead of guide tubes to center and stabilize the device allows for greater stability because an inflated balloon is able better anchor the device in place as opposed to guide tubes. The use of a balloon also reduces the pressure applied to the lumen walls because the surface area in contact with the lumen is much greater as opposed to when guide tubes are used, reducing the risk of damaging the lumen wall. By using a balloon, the device can also be used in vessels which are non-circular, whereas this would not be possible with the Peregrine System™ because the Peregrine system is centered in the vessel using the needle guide tubes. Many veins are non-circular in nature and the use of guide tubes to center and stabilize the device in such vessels would not be optimal because it would place excess pressure on certain points on the vessel wall. The current invention, however, can be used in such vessels as the balloon would force the vessel into a circular shape for treatment to be optimal.

### BRIEF DESCRIPTION OF THE FIGURES

FIG. 1 is a schematic view of a first embodiment of the invention with the sheath in the extended position.

FIG. 2 is a schematic view of a first embodiment of the invention with the sheath in the retracted position, the balloon in the deflated state, and the needles in the retracted position.

FIG. 3 is a schematic view of a first embodiment of the invention with the sheath in the retracted position, the balloon in the inflated state, and the needles in the retracted position.

FIG. 4 is a schematic view of a first embodiment of the invention with the sheath in the retracted position, the balloon in the inflated state, and the needles in the extended position.

FIG. 5 is an enlargement of the distal region of the device in FIG. 2.

FIG. 6 is an enlargement of the distal region of the device in FIG. 3.

FIG. 7 is a transverse cross section to highlight the three guide tubes of the device in FIG. 3.

FIG. 8 is an enlargement of the distal region of the device in FIG. 4.

FIG. 9 is a transverse cross section to highlight the three guide tubes and needles of the device in FIG. 4.

FIG. 10 is a schematic view of an example of a handle.

FIG. 11 is a schematic view of an example of a possible support structure.

FIG. 12 is a schematic view of an example of another possible support structure.

FIG. 13 is a transverse cross section of the inflated balloon and flexible tip.

FIG. 14 shows the distal region of a second embodiment of the invention with the balloon in the deflated state, two guiding cannula, and the needles in the retracted position.

FIG. 15 shows the distal region of a second embodiment of another embodiment of the invention with the balloon in the inflated state, two guiding cannula, and the needles in the extended position.

FIG. 16 shows a cross section of the guiding cannula with a curve at the distal terminal end.

FIGS. 17 to 21 show possible cross sections of the catheter body. The embodiments shown in FIGS. 17, 18 and 20 do not require a guidewire, while those of FIGS. 19 and 21 require a guidewire for use.

### DETAILED DESCRIPTION OF PARTICULAR EMBODIMENTS

The invention provides a drug delivery device, i.e. a catheter (e.g. a rapid exchange catheter or an over the wire catheter), that can be inserted into a vessel or lumen within the body and used to administer drugs to tissue surrounding the vessel or lumen. The drug delivery device comprises:
an axially extending elongate member having a proximal portion, a distal portion, a first lumen and a second lumen;
an inflatable balloon coupled to the distal portion of the axially extending elongate member, the inflatable balloon having a proximal end and a distal end that are coupled together by a working portion, the balloon also having an exterior surface and an interior surface that defines a balloon chamber, where the first lumen of the axially extending elongate member is in fluid communication with the balloon chamber;
two or more guiding cannulas, each having a proximal portion and a distal portion, where the proximal portion of each guiding cannula is coupled to the axially extending elongate member,
two or more needles, each having a lumen and being housed within one of the two or more guiding cannulas, such that each needle is reversibly extendable from the distal portion of the guiding cannula that houses it, and where the second lumen of the axially extending elongate member is fluidly connectable to the lumen of each needle, wherein,
when the inflatable balloon is inflated, the distal portion of each of the two or more guiding cannulas do not extend beyond an outermost diameter of the exterior surface of the inflatable balloon.

In some embodiments, the drug delivery device may comprise an inflatable balloon coupled to the distal end of the axially extending elongate member. The device may further include a balloon support element within the inflatable balloon (such as a wire or other element to prevent axial compression of the balloon), which may be coupled to the distal portion of the axially extending elongate member and to the distal end of the inflatable balloon.

In some embodiments, the inflatable balloon may be disposed over the distal portion of the axially extending elongate member. For example, the inflatable balloon may be coupled to the distal end of the axially extending elongate member and disposed over the distal portion of the axially extending elongate member.

In some embodiments, the axially extending elongate member may comprise a third lumen, which may be suitable for mounting the device on a guide wire. This may advantageously facilitate the navigation of the device to a target site within the blood vessel network.

The two or more guiding cannulas may be coupled to the device proximal to the balloon and positioned around the circumference of device at substantially evenly spaced intervals.

In some embodiments, the distal portion of the guiding cannulas may not be affixed to the inflatable balloon, and/or may be able to move freely with respect to the inflatable balloon. As explained herein, this allows the distal ends of the guiding cannulas to adopt a configuration/position inside a vessel that allows the needles to pierce the vessel at an advantageous angle, such as substantially perpendicular to the vessel wall. In some embodiments, the distal portion of the guiding cannulas may be configured to contact the exterior surface of the inflatable balloon and move with the exterior surface of the inflatable balloon as the inflatable balloon is inflated. This also assists in enabling the distal ends of the guiding cannulas to adopt a configuration/position inside a vessel that allows the needles to pierce the vessel at an advantageous angle, such as substantially perpendicular to the vessel wall.

Nevertheless, where a guiding cannula is configured to contact the exterior surface of the inflatable balloon and move with the exterior surface of the inflatable balloon as the inflatable balloon is inflated, the guiding cannula may also be configured to at least partially revert back to its original shape or configuration once the inflatable balloon is deflated. This ensures that the device may easily be removed from the vessel after use without the cannulas damaging the vessel walls, and for similar reasons ensures that the device may be reinserted to a vessel without the cannulas damaging the vessel walls.

The inflatable balloon may comprise a proximal cone portion, a central cylindrical portion and a distal cone portion. In such embodiments, the distal portions of the guiding cannulas may be configured to contact the exterior surface of the proximal cone portion of the inflatable balloon and move with the exterior surface of the proximal cone portion. Thus, the guiding cannulas may be moved by the inflation of the balloon, such that the guiding cannulas adopt a position ready for the needles housed within to pierce the vessel wall when the balloon is fully inflated.

The device may further comprise support elements located at the proximal portion of each guiding cannula, so as to fix it in place and whilst allowing the distal portion of the guiding cannula to move and adopt the desired orientation during use. The distal tips of these guiding cannulas may be curved outwardly and terminate along the proximal cone of the balloon. This means that the distal tips of the guiding cannulas do not extend (i.e. in the radial direction) beyond an outermost diameter of the exterior surface of the balloon, so they do not contact the vessel walls during navigation of the device to a target site. Such contact between the cannulas and vessel walls during navigation could lead to damaging scraping of the vessel walls.

The guiding cannulas may comprise slits cut into one side of the guiding cannulas. These slits may serve to control the direction in which the guiding cannulas move or deform as the balloon is inflated.

In some embodiments, the needles may comprise a curved end portion. This advantageously allows each needle to be configured such that when the needle is extended from the cannula that houses it, the curved end portion curves outwardly in the radial direction from the axially extending elongate member. This configuration allows the needle to pierce the vessel at an angle that is closer to perpendicular than would be possible with a straight needle—thereby allowing a greater penetration depth to needle advancement ratio.

In such embodiments where the needles comprise a curved end portion, each needle may be configured such that when the needle is extended from the cannula that houses it, the curved end portion curves outwardly in the radial direction from the axially extending elongate member.

In some embodiments, the two or more guiding cannulas may comprise a curved portion at their distal ends. This may serve to bend an otherwise straight needle as it is extended from the guiding cannula, or may serve to facilitate the extension of a curved needle from the guiding cannula.

In some such embodiments, the two or more guiding cannulas may each comprise an opening facing outwards from the catheter at their distal ends, which may also serves to facilitate the bending and/or direction of a needle extended from the guiding cannula.

In some embodiments, the guiding cannulas may be made from a metal or alloy. This may be especially advantageous when the needle comprises a curved end portion, since the needle tip may contact the edge of the guiding cannula, and a plastic cannula may be pierced or damaged by a curved needle. The use of a metal or alloy for the guiding cannula may also be advantageous when it is desirable to slightly deform the needle during extension or retraction. For example, a straight needle may be used with a curved cannula to achieve a needle penetration angle that is closer to perpendicular than could be achieved with a straight cannula.

This may be advantageous since straight needles may be easier or cheaper to procure. Alternatively, a curved needle may advantageously be used with a straight cannula when it is desirable for the device to have a low profile in the needle retracted state (which may be achieved with a straight cannula), yet still obtain the benefits of a curved needle when it comes to the penetration angle of the needle.

In some embodiments, the device may comprise a sheath movable disposed over the axially extending elongate member. The sheath may serve to house the guiding cannulas and other device components during navigation of the device to and from a target site to be treated. This helps to avoid damaging the device or vessel walls during navigation.

In some embodiments, the sheath may have an extended state and a retracted state, where in the extended state the sheath covers the entirety of the two or more cannulas and at least part of the inflatable balloon, and in the retracted state the sheath does not cover a part of the inflatable balloon. In some embodiments, in the retracted state the sheath covers only part of the two or more cannulas, since this may allow for greater flexibility in needle position during use, but may still provide the benefits discussed above.

The needles in the device of the invention may be simultaneously reversibly extendable, and/or may be independently reversibly extendable.

In some embodiments of the invention, the second lumen of the axially extending elongate member may comprise two or more sub-lumens, each sub-lumen being fluidly connectable to the lumen of one of the two or more needles. In some such embodiments, the two or more sub-lumens are in some cases not in fluid communication with each other. This allows for different liquids (e.g. different active agents) to be administered via the different needles.

In some embodiments, the device may comprise a support structure for positioning and securing the guiding cannulas to the axially extending elongate member. This may serve to control the degree of movement possible for the distal ends of the guiding cannulas and assist in more accurately controlling the site of needle penetration of the vessel wall.

In some embodiments, the distal end (i.e. the distal tip) of the axially extending elongate member may be coupled to, or comprise, a flexible tip. This may help to avoid damaging the vessel wall during navigation of the device to a site to be treated.

In some embodiments, the device may comprise at least one radiopaque element, which will help to inform an operator as to the location of the device within a patient's body. In such embodiments, the radiopaque element may be positioned within the balloon chamber, on the axially extending elongate member, on a guiding cannula, and/or on a needle.

A handle may be located at the proximal end of the device. The handle may comprise one or more of the following:
a mechanism for controlling the maximum needle extension depth;
a visual indicator of needle extension depth (e.g. a visual indicator of current needle extension depth so that an operator knows the current state of the needles);
a means for reversibly extending the needles from the cannulas (e.g. a first means for roughly reversibly extending the needles and a second means for finely reversibly extending the needles);
a means for extending and retracting the sheath;
an inflation port for the balloon (i.e. an inflation port fluidly connected to the first lumen of the axially extending elongate member); and
an inflation port for the needles (i.e. an inflation port fluidly connected to the second lumen of the axially extending elongate member).

As will be appreciated by a person skilled in the art, these features may beneficially assist a user in operating the device of the invention in a safe and efficient manner.

In some embodiments of the invention, the device may be a rapid exchange or over the wire catheter.

In a specific embodiment of the present invention:
each needle may comprise a curved end portion;
the distal portions of the guiding cannulas may be configured to contact the exterior surface of the inflatable balloon and move with the balloon exterior as the balloon is inflated; and
the guiding cannulas may be able to at least partially revert back to their original shape after the balloon is deflated.

In a further example of this embodiment, each needle may be configured such that when the needle is extended from the cannula that houses it the curved end portion curves outwardly in the radial direction from the axially extending elongate member.

Further details of the device of the invention and the use of the device are described below.

In use, the device may be first tracked into location with the balloon deflated and the sheath (when present) positioned over at least part of the balloon and guiding cannula, with the needles completely within the guiding cannula. Once it is confirmed that the device is in place, the sheath may be retracted, balloon inflated and needles advanced outwardly to the desired depth. The inflation of the balloon pushes the distal portion of the guiding cannula outwards. The inflated balloon also centers the device within the vessel, while anchoring it in place. The inflated balloon additionally serves as a stable platform for the guiding cannulas as they rest on the proximal cone of the balloon. This provides the most optimal condition for the needles to advance outwardly from the guiding cannula into the vessel wall. When the needles are in place, a therapeutic agent may be introduced through the needles for treatment. After treatment, the needles may be retracted, balloon deflated and the device removed.

The components of the device according to the invention are described in more detail below.

The inflatable balloon may be used to anchor the device within a lumen when the balloon is inflated such that the wall of the balloon contacts the inner wall of the lumen. Typically, the inflatable balloon may be coupled to the distal end of the axially extending elongate member, and the inflatable balloon may be disposed over the distal portion of the axially extending elongate member. Typically, the balloon may be a low pressure balloon, i.e. it is designed to be inflated to a comparatively low pressure, to avoid causing damage to the lumen/vessel. For example, the balloon may typically be inflated to a pressure of 3 atmospheres or less, such as about 2 atmospheres. During use, the balloon may be inflated from a collapsed (uninflated) configuration to an expanded (inflated) configuration. The balloon may be capable of being deflated after inflation to return to the collapsed/deflated configuration, so that the catheter may be moved to another target site or removed from the body. The inflatable balloon may have a proximal cone portion, a central cylindrical portion and a distal cone portion.

In some embodiments of the invention, the axially extending elongate member may comprise a third lumen, which may be suitable for mounting the device on a guidewire. In the embodiments of the catheter in which a guidewire lumen is not present, there can be a risk of the balloon crumpling or folding in on itself as the catheter is moved distally within the lumen if there is no support for the balloon. Thus, when the device (catheter) does not comprise a guidewire lumen, it may comprise a support element (such as a support wire) disposed within the balloon. The support element (e.g. support wire) may be coupled to the catheter at two locations, one being distal to the balloon and one proximal to the balloon. For example, a support element may be coupled to the distal portion of the axially extending elongate member and to the distal end of the inflatable balloon.

The device comprises two or more guiding cannulas, such as 2, 3, 4, 5 or 6 guiding cannulas, each of which houses, or is configured to house, an extendable needle. Typically, the device comprises from 2 to 4 guiding cannulas, such as 2 or 3 guiding cannulas. The guiding cannulas are typically disposed around the circumference of the axially extending elongate member at substantially evenly spaced intervals, to ensure even administration of a drug to the tissue surrounding a lumen/vessel. The guiding cannulas are typically made from a hard and strong material, for example one or more metals or alloys. A hard and strong material may be used so that the cannula is rigid and able to bend or otherwise deform/shape a needle that is placed within the cannula and/or extends from the cannula. For example, the cannula may be straight and able to deform a curved needle such that the needle can be housed within the cannula. Alternatively, the cannula may comprise a bent portion that is able to deform a straight needle that is extended through the curved portion out of the cannula, such that the curved portion of the cannula imparts a curve into the needle. The guiding cannula may have its proximal end connected to the catheter body, with the distal region free and terminating along or adjacent to the balloon proximal cone. Generally, the guiding cannulas may be arranged around the catheter at evenly spaced intervals. The distal terminal opening of the guiding cannula are typically configured such that the open ends are all facing perpendicularly outwardly from the central axis of the catheter. In some embodiments, the distal end of the guiding cannula may be terminated with a curved portion, which advantageously imparts a curve into a needle as the needle extends from the guiding cannula. In other embodiments, the guiding cannula may be straight.

In some embodiments of the invention, the distal portion of each guiding cannula may be able to move freely with respect to the inflatable balloon, and/or may be not affixed to the inflatable balloon. The distal portions of the guiding cannulas may be configured to contact the exterior surface of the inflatable balloon and move with the exterior surface of the inflatable balloon as the balloon is inflated. This means that inflation of the balloon will place the cannulas in the desired position for extension of the needles. In this case, the guiding cannulas are typically configured to at least partially revert back to their original shape when the inflatable balloon is deflated. In an alternative configuration, the guiding cannulas may terminate proximal to the inflatable balloon, such that the position of the guiding cannulas is unaffected by inflation and deflation of the balloon.

The guiding cannulas may be positioned such that their distal ends typically terminate before, or proximal to the proximal region of the balloon. This means that the cannulas are only displaced by the proximal region of the balloon, which typically has a cone shape (i.e. a proximal cone portion). As such, when the balloon is fully expanded, the distal ends of the guiding cannulas will not extend in the perpendicular direction beyond the outermost diameter of the balloon. This is important to avoid the guiding cannulas impacting the wall of a vessel/lumen during use, since such an impact could cause damage to the vessel or lumen.

The guiding cannula may be supported by a support structure. The support structure is for positioning and securing the guiding cannulas to the axially extending elongate member. Thus, the support structure may hold the proximal and middle portions of the cannula in place, but nonetheless allow the distal portion to bend outwardly in response to the inflation of the balloon. Alternatively, the support structure may support and hold the entirety of the guiding cannula in place. In this case, the guiding cannula(s) may be housed within the support structure. The guiding cannulas are at least partially able to revert back to their initial configuration after the balloon is deflated.

Each guiding cannula houses, or is configured to house, a needle, which is movably disposed within the lumen of said guiding cannula, such that the needle has an extended state and a retracted state. The terminal end of each needle can be curved laterally in the outward direction from the catheter (i.e. each needle may comprise a curved end portion). Thus, the needles are typically configured such that when the needles are extended from the cannulas that house them, the curved end portion of each needle curves outwardly in the radial direction from the axially extending elongate member, Generally, the needles have a larger radius of curvature than the guiding cannula they are housed in. In this case, when a needle transitions from the retracted state to the extended state, the distal terminal bend of the guiding cannula forces the needles to adopt a curvature with a smaller radius. The needles are coupled to a delivery cannula allowing the needles to be in fluid communication with each other and the proximal end of the catheter. The needles may be simultaneously reversibly extendable, and/or independently reversibly extendible.

The needles each comprise a lumen that is fluidly connectable to a lumen (e.g. the second lumen) within the axially extending elongate member, such that fluid can be delivered to the needles along the axially extending elongate member. The second lumen of the axially extending elongate member may comprise two or more sub-lumens, with each sub-lumen being fluidly connectable to the lumen of a needle. Typically, the two or more sub-lumens are not in fluid communication with each other.

A flexible tip may be disposed at the distal end of the device, coupled to the distal end of the axially extending elongate member. A handle is typically disposed at the proximal end of the device. The handle may be configured such that it is able to manipulate needles from the extended state to the retracted state and vice versa. The handle may comprise markings to determine the displacement of the needles in order to assist in controlling the penetration depth of the needles into the lumen wall and surrounding tissue. The handle may also comprise a mechanism to set the maximum penetration depth when the needles are advanced. Such a mechanism is typically lockable and unlockable, such that the mechanism can be easily and quickly unlocked if the needles needs to be advanced further. The handle may comprise means such as using threads to turn rotational movement into lateral displacement, to reduce the force required for the displacement of the needles.

The device may comprise at least one radiopaque element, i.e. an element visible under fluoroscopy or other imaging method. The at least one radiopaque element may be disposed in a location which aids in positioning of the catheter, for example, within the balloon chamber, on the axially extending elongate member, on a guiding cannula, and/or on a needle. The flexible tip, when present, may also comprise a radiopaque element. Thus, an appropriate imaging technique may be used while the catheter is being navigated to a target site within a body, to provide accurate real-time information regarding the location of the catheter within the body.

The device may comprise a sheath movably disposed over the axially extending elongate member. The sheath is typically movably disposed over the part of the extending elongate member onto which the guiding cannulas are disposed, such that the sheath is movably disposed over the guiding cannulas. The sheath typically has an extended state and a retracted state, and may in some embodiments be manipulated via the handle. In the extended state, the distal end of the sheath may be distal to the distal end of the guiding cannula, such that the sheath covers the entirety of the guiding cannulas and at least part of the inflatable balloon. In the retracted state, the distal end of the sheath is typically proximal to the proximal cone of the balloon, such that it does not cover a part of the balloon. In the retracted state, the sheath typically covers only part of the cannulas. As such, in the extended state the sheath may cover the cannula and protect the vessel/lumen walls from the cannula during navigation along the vessel. In the retracted state the cannula are exposed to the interior of the vessel such that the needles may be extended and penetrate the wall of the vessel. In one variant, the sheath may be a flexible sheath that covers the proximal cone of the balloon, and upon inflation of the balloon the sheath will naturally retract due to the pressure imparted on the sheath from inflation of the balloon. In another variant, the sheath may be rigid and cover the distal end of the guiding cannulas, but the sheath may comprise weak points located over the guiding cannulas. During tracking of the catheter to the target site, the rigid sheath will be over the distal end of the guiding cannulas but when the balloon is inflated, the expansion in diameter of the balloon will result in the rigid sheath tearing at the weak points, thus exposing the distal end of the guiding cannulas to allow for unobstructed needle deployment.

The device may comprise a handle disposed at the proximal end of the axially extending elongate member, which may serve a number of different purposes. The handle may house both an infusion and inflation luer, which allow for the infusing for substances through the needles and inflation of the balloon respectively. If a sheath is present, the handle may also house a flushing luer which allow the flushing of the space within the sheath. The handle may be responsible for the manipulation of the needles from the retracted to the extended state and vice versa. It is preferable to have fine control when manipulating the needles to the desired depth, since this helps to avoid damaging the tissue and also ensures that the drug is infused to the correct tissue. The handle may comprise a mechanism for controlling the maximum needle extension depth. The handle may also comprise a means for reversibly extending the needles from the cannulas, which means may comprise a first means for roughly reversibly extending the needles and a second means for finely reversibly extending the needles. Means such as gears or screws can be incorporated into the handle to provide a fine control over the extension and retraction of the needles, as well as the maximum extension depth. Such means may also allow for less force to be used to displace the needles. The handle may comprise markings to help a user to determine the needle displacement relative to the guiding cannula as they are being extended. The handle may also be responsible for determining or setting the fully extended and retracted states. The fully retracted state of the needles may be achieved when the needles are unable to retract further. The fully retracted state is important in the embodiments in which curved needles are used with a guiding cannula with a free distal end and where a sheath is not present, since the needles must be retracted sufficiently far that they do not cause the guiding cannula to curve outwards. A pre-determined maximum extended state may be important since it may allow a user to avoid damaging sensitive tissue by over-extending the needles.

In an embodiment of the invention:
each needle comprises a curved end portion;
the distal portions of the guiding cannulas are configured to contact the exterior surface of the inflatable balloon and move with the balloon exterior as the balloon is inflated; and
the guiding cannulas are able to at least partially revert back to their original shape after the balloon is deflated, optionally wherein each needle is configured such that when the needle is extended from the cannula that houses it the curved end portion curves outwardly in the radial direction from the axially extending elongate member.

The invention is described in further detail below with particular reference to the Figures.

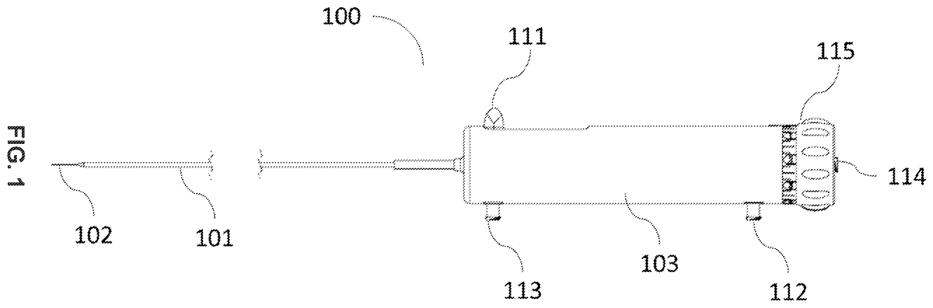

FIG. 1 to FIG. 4 show a device according to a first embodiment of the invention. FIG. 1 to FIG. 4 are side views of a device 100 in different stages of use. In FIG. 1, the device 100 is in the sheathed state with the sheath 101 fully extended. A sheath 101 protects the walls of the lumen when the device is tracked through it. Located at the distal portion of the device is a flexible tip 102 which allows the device 100 to have improved trackability through the lumen. A handle 103 of the device houses a slider 111 which is used to control the displacement of the sheath 101. The device also comprises an inflation luer 112 (used to inflate the inflatable balloon 121), a flushing luer 113 (used to flush the inside of the sheath 101), an injection luer (114 which allows fluid access the needles 123), a nut 115 (to control the displacement of the needles 123), and a bolt 116 (which is used to displace the needles 123). FIG. 1 shows the state the device 100 will be in when it is being tracked into position for treatment. After the device 100 has been tracked into position, sheath 101 will be retracted.

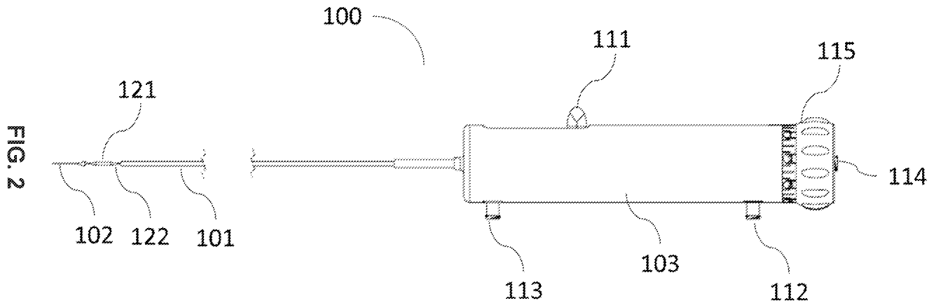

FIG. 2 shows the device with the sheath 101 in a retracted state, after the slider 111 was advanced proximally to the retracted position. The retraction of the sheath exposes the balloon 121 and guiding cannula 122. After the sheath 101 is in the retracted position, the balloon may be deployed (inflated).

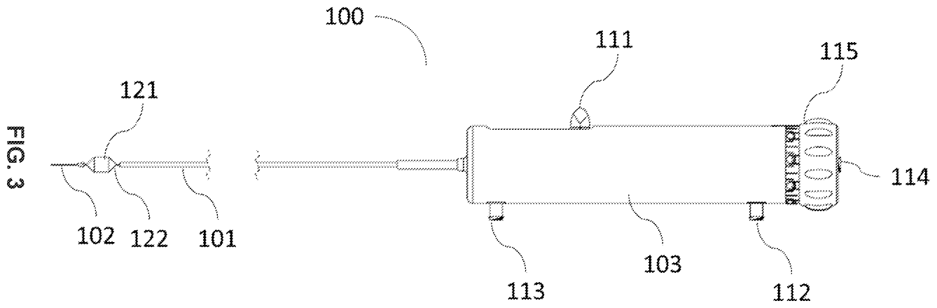

FIG. 3 shows the device 100 when the balloon 121 is inflated. The inflated balloon 121 will anchor the catheter to the vessel wall and also center the catheter within the lumen. The inflation of balloon 121 also causes the distal end of the guiding cannula 122 to be displaced outwardly. Once the device is in position and secured, the nut 115 can be rotated relative to the handle 103 in order to advance the needles 123 forward. FIG. 4 shows the configuration of the device with the needles 123 extended.

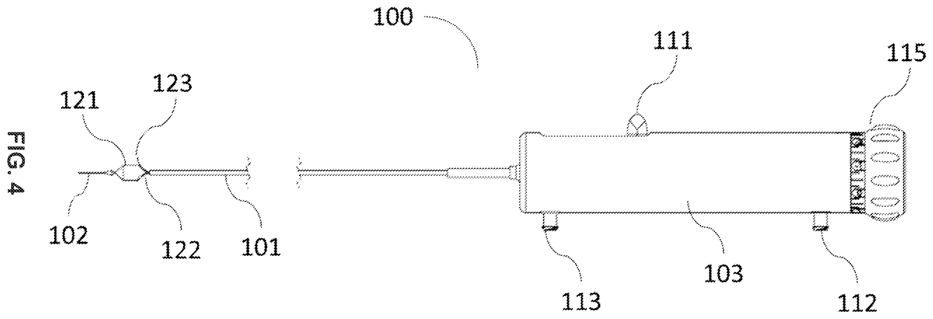

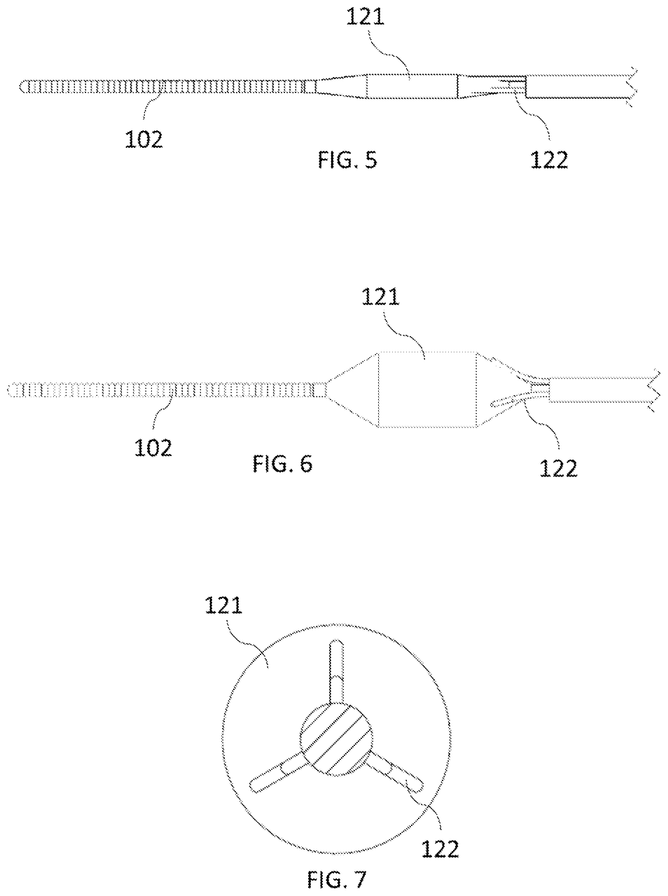

FIG. 5 to FIG. 9 show an expanded view of the distal portion of the device of the first embodiment of the invention in greater detail in the different stages of use. FIG. 5 shows the distal portion of the device configuration as shown in FIG. 2. When the balloon 121 is deflated, the guiding cannula 122 is mostly straight and it is possible for the distal ends of the guiding cannula 122 to be within the folds of the balloon 121. FIG. 6 shows the shows the distal portion of the device configuration as shown in FIG. 3 when the balloon 121 is inflated. In the inflated state, the guiding cannula 122 comes into contact with the balloon 121 which pushes its distal end causing it to bend outwardly. FIG. 7 shows the transverse view of the distal end of the device 100 in the configuration of FIG. 3 when the balloon 121 is viewed from the proximal end. It is important that the guiding cannula 122 does not extend past the outermost diameter of the balloon 121. This is because the cannula would contact the walls of the lumen, possibly causing damage during inflation of the balloon. In addition, the cannula would prevent the balloon from coming into direct contact with the walls of the lumen, resulting in poor anchoring of the device.

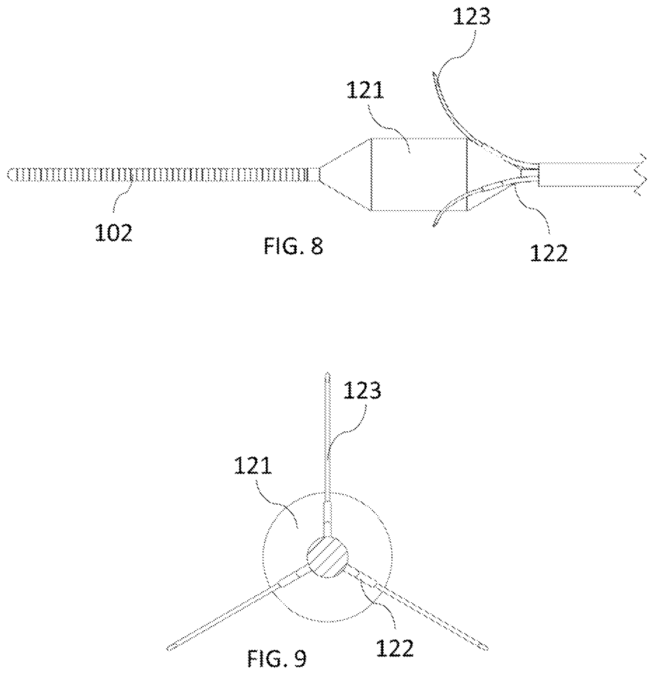

FIG. 8 shows the shows the distal portion of the device configuration as shown in FIG. 3 when the balloon 121 is inflated and the needles 123 are extended. It is preferable for the exposed portions of the needles to be curved outwardly in the same direction as the distal end of the guiding cannula 122 as this allows for greater penetration depth and more control over the penetration depth. FIG. 9 shows the transverse view of the distal end of the device 100 in the configuration of FIG. 4 when the balloon 121 is viewed from the proximal end.

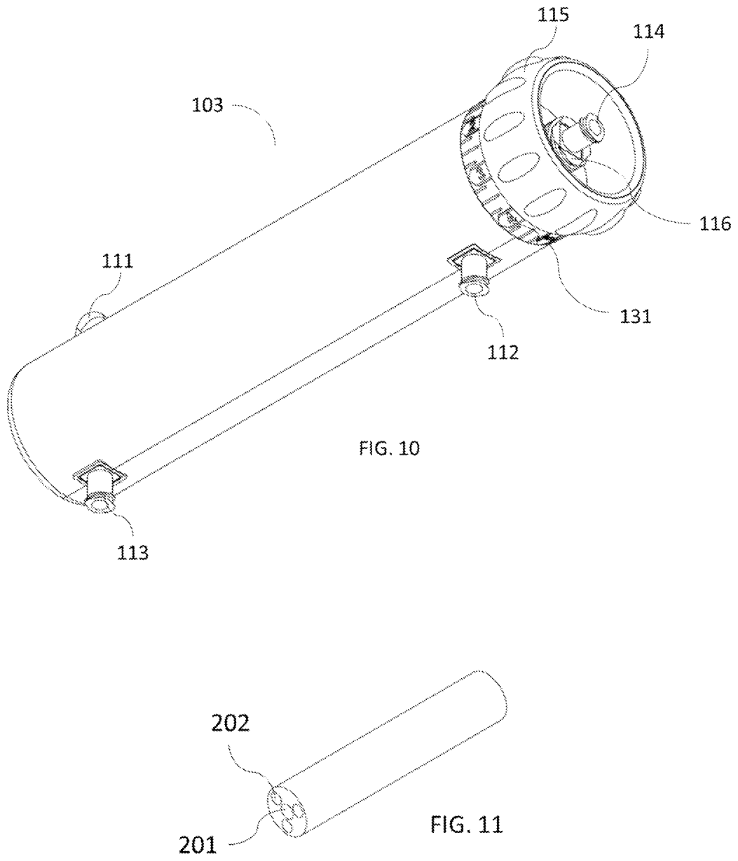

FIG. 10 shows a schematic view of an example of a handle 103. In this example, the proximal end of the handle is responsible for the movement of the needles. When the nut 115 is rotated, the treads located within will engage the threads located on the bolt 116, which is connected to the needles 123, allowing for movement. The markings 131 are then used to determine the penetration depth of the needles 123.

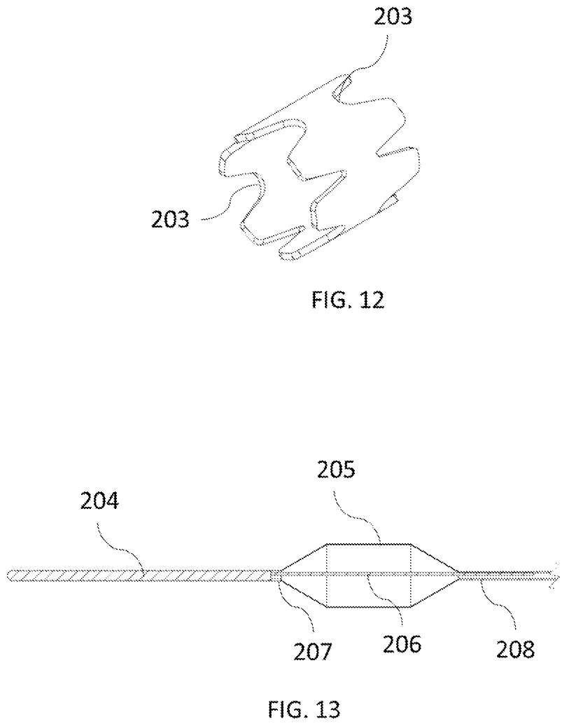

FIG. 11 and FIG. 12 show examples of support structures. The support structure ensures that the guiding cannulas are in the correct position on the catheter. The support structure of FIG. 11, has four lumens, the central lumen 201 is to house any part of the catheter which is central to the device, such as the inflation tube coming out the proximal end of the balloon. In the support structure of FIG. 11, there are three outer lumens 202 which are equally spaced around the central lumen 201 which can be used to house the guiding cannulas. The support structure shown in FIG. 11 is the type that is meant to be placed proximal to the balloon. The support structure of FIG. 12 has grooves 203 which are evenly spaced and inside which a guiding cannula can rest. The support structure of FIG. 12 can be positioned on the proximal cone of the balloon in order to provide support at a more distal location. The support structures can be use independently but when used together, they may show a synergistic effect in securing the orientation of the guiding cannulas.

FIG. 13 shows a cross section of a possible construction of the balloon region. In some embodiments of the device, a guide wire will not be used. A guidewire may be used in conventional balloon catheters to support the soft balloon. In embodiments of the invention that do not require a guidewire for use, a support wire 206 may be used to support the balloon 205. The support wire 206 is coupled to both the distal balloon leg 207 and proximal balloon leg 208. A flexible tip 204 may be coupled to the distal end of the balloon to improve the trackability of the device.

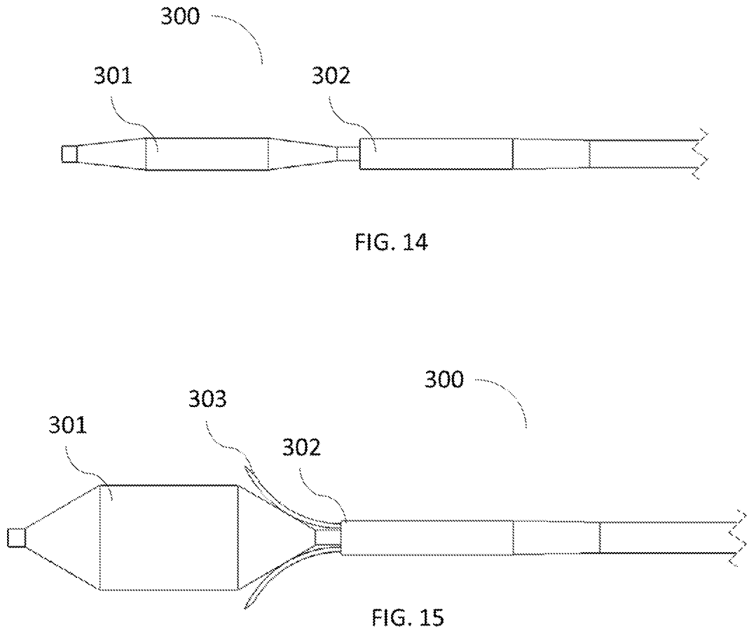

FIG. 14 and FIG. 15 show a device according to a second embodiment of the invention. These Figures show the distal region of device 300 in different stages of use. In the device 300 according to the second embodiment, multiple guiding cannulas (not shown) are housed within a support structure 302 that is fixed over the elongate member. The guiding cannulas do not comprise any “free” portions that move with the balloon, Instead, the guiding cannulas each terminate before the balloon 301 and therefore do not come into contact with the balloon 301. FIG. 14 shows device 300 when the balloon 301 is in the deflated state and the needles 303 are in the retracted position. This is the state in which the device 300 will be tracked into position. FIG. 15 shows device 300 when the balloon 301 is inflated and the needles 303 are extended. Since the guiding cannulas terminate before the balloon 301, it is crucial that the needles 303 exiting the guiding cannula have a radius of curvature that is sufficiently small so that it does not puncture the inflated balloon 301.

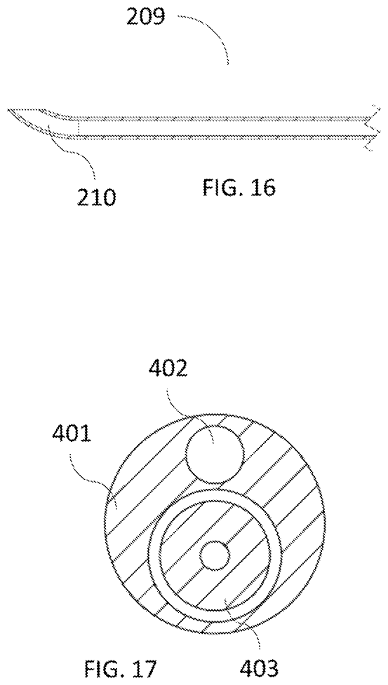

FIG. 16 show the distal end of a guiding cannula 209 which has a distal end which terminated in a curve 210. In order to keep the profile of the device small, the curve 210 should be short so that the profile of the guiding cannula 209 does not increase significantly. The terminal curve 210 is beneficial as it allows the needle exiting of the guiding cannula to exit at an angle. Also, the curve 210 will cause the needle exiting the guiding cannula 209 to deform into a curved shape. Both of which are beneficial to various embodiments of the device.

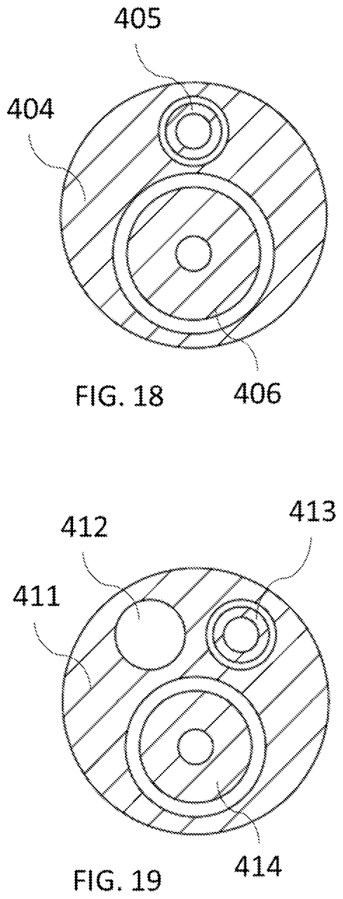

FIG. 17 and FIG. 18 show the cross sections of two possible constructions of the catheter body in the embodiment of the device which does not require a guide wire. Although a sheath is not depicted in both figures, it is possible for a sheath to be present over the depicted constructions. FIG. 17 shows a construction of the body with two components, a two lumen tube 401 and an injection tube 403, The two lumen tube 401, houses an inflation lumen 402 (i.e. first lumen) as well as the injection tube 403 comprising the second lumen. The injection tube 403 is able to move relative to the two lumen tube 401 which allows the advancement and retraction of the needles. The two lumen tube 401, is also connected directly to the balloon which allows the inflation and deflation of the balloon through the inflation lumen 402. FIG. 18 shows a construction of the body with three components, a two lumen tube 404, an injection tube 406 and an inflation tube 405. The construction depicted in FIG. 18 is similar to that of FIG. 17 but the two lumen tube 404 is not connected directly to the balloon. In FIG. 18, the inflation tube 405 is connected directly to the balloon for inflation and deflation. The inflation tube 405 is also not able to move relative to the two lumen tube 404.

FIG. 19 show the cross section of a possible construction of the catheter body in the embodiment of the device which requires a guide wire. A three lumen tube 411 houses the inflation lumen 412 (i.e. first lumen), injection tube 414 comprising the second lumen, and the guidewire tube 413. Similar to FIG. 17 and FIG. 18, a sheath can be present and the injection tube 414 is able move relative to the three lumen tube 411 to control the needles. The balloon can also either be connected directly to the three lumen tube 411 for inflation and deflation via the inflation lumen 412 or be connected to a separate inflation tube which is housed in the inflation lumen 412 similar to what is depicted in FIG. 18.

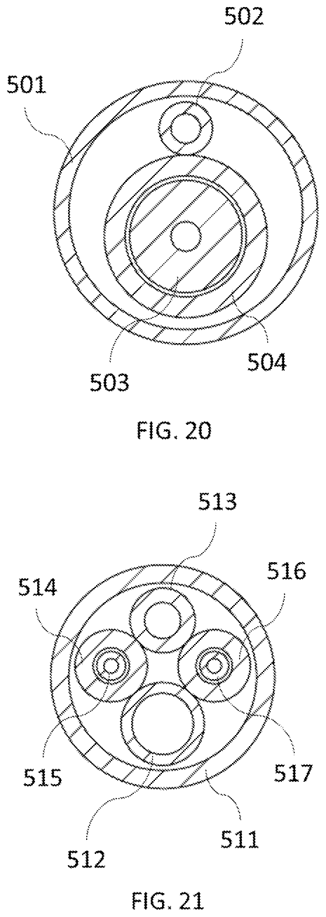

FIG. 20 and FIG. 21 show the cross sections of two possible constructions of the catheter body in an embodiment of the device which has a sheath. FIG. 20 show the cross section of a possible construction of the catheter body in the embodiment of the device which does not require a guide wire and is composed of a sheath 501, an inflation tube 502 comprising the first lumen, an injection tube 503 comprising the second lumen, and a body tube 504. The injection tube 503 is housed within and able to move relative to the body tube 504. The sheath 501 houses the inflation tube 502 and body tube 504 and is able to move relative to them. The inflation tube 502 and body tube 504 can be held together by adhesives or by using a heat shrink tube around them.

FIG. 21 show the cross section of a possible construction of the catheter body in the embodiment of the device which requires a guide wire and is composed of a sheath 511, a guidewire tube 512, an inflation tube 513 comprising the first lumen, two body tubes 514 and 516, and two needles 515 and 517. The embodiment of the device in FIG. 21 is one in which the needles are independent of each other and where the second lumen comprises two sub-lumens formed within the body tubes 514 and 516. The needles 515 and 517 are housed within and are able to move relative to the body tubes 514 and 516 respectively, each of which comprise one of the sub-lumens of the second lumen. The sheath 511 houses the guidewire tube 512, inflation tube 513, and both body tubes 514 and 516 and is able to move relative to them. The guidewire tube 512, inflation tube 513, and both body tubes 514 and 516 can be held together by adhesives or by using a heat shrink tube around them.

A first embodiment of the invention is described above with reference to FIG. 1 to 9. The device according to the first embodiment comprises three guiding cannula that have a distal end terminating at the proximal cone of the balloon, adjacent to the proximal end of the working length of the balloon, such that the guiding cannula bend outwardly in response to inflation of the balloon. Each guiding cannula houses a retractable needle that is curved laterally at the distal end in the direction outwards from the catheter. The guiding cannula are spaced evenly around the catheter, and supported by a support structure. The support structure holds or supports the proximal and middle regions of the cannula, preventing unwanted movement, but allows the distal end of the cannula to move in response to expansion of the balloon. The device comprises support wire that supports the balloon, helping to prevent collapse or folding of the balloon, and a flexible tip in order to improve the trackability of the device. Although not shown, a skilled person will appreciate that the device of the first embodiment may comprise a retractable sheath that covers the cannula during navigation along a vessel to a target site, as described in more detail herein. In some cases, the first embodiment comprises a sheath that covers the balloon and guiding cannulas.

A second embodiment of the invention is described above with reference to FIGS. 14 and 15. In this embodiment, the device comprises two guiding cannula that terminate before the balloon (i.e. terminate proximal to the balloon), such that the guiding cannula are not impacted by the balloon when the balloon inflates. This means the distal ends of the guiding cannula are not displaced by inflation of the balloon, and so the needles must have a curvature that allows them to extend from the cannula and not pierce the balloon. This can be achieved by using needles that have a natural curvature, and which will adopt a curved conformation once they are no longer restrained by the cannula. Alternatively, the cannula may comprise a curved distal portion that imparts a curve onto a needle as it extends from the cannula. The cannulas are supported by a support structure, but in this embodiment the distal end of the cannula does not need to move in response to expansion of the balloon, and so the support structure may hold the entire cannula in place. In the embodiment shown, the device does not comprise a flexible tip, although a skilled person would appreciate that a flexible tip could readily be included on this embodiment. The device comprises a cannula/lumen for the passage of a guide wire through the device. In some cases, the second embodiment comprises a sheath that covers the balloon and guiding cannulas.

A third embodiment of the invention corresponds to the first embodiment, except that it does not comprise a support wire or flexible tip, but instead comprises a lumen or cannula for the passage of a guide wire. The third embodiment also comprises two or more guiding cannulas.

A fourth embodiment of the invention corresponds to the first embodiment, except that it comprises two guiding cannulas, each housing a needle, rather than three cannulas/needles. The specific embodiments described above and in the Figures (such as the first, second, third and fourth embodiments) may be modified to include any other features disclosed herein. For example, each of the specific embodiments may be modified to include a sheath that covers the balloon and guiding cannula(s). All embodiments disclosed herein may be modified either to comprise a cannula/lumen for passage for a guidewire; or to comprise a support wire preferably in combination with a flexible tip. Typically a device will not comprise both a support wire and a lumen for a guidewire, since the use of a guidewire would make a support wire redundant. In general, a flexible tip is preferred when a support wire is used (since the support wire does not guide the passage of the device but merely supports the structure of the balloon), but a flexible tip is not necessary when a guidewire is used since the guidewire guides the passage of the device through a vessel. Finally, any of the first to fourth embodiments may comprise a radiopaque element.

In general, a device that is intended for use inside larger vessels may comprise a support wire and a flexible tip, while a device intended for use inside smaller, more peripheral vessels, may be used in conjunction with a guidewire to help navigation through the narrower vessels. In addition, a device intended for use in smaller vessels may comprise a lower number of needles, such as two needles, and the guiding cannula for these needles may have a lower profile than those intended for use in large vessels. For example, in a device intended for use in smaller vessels, the guiding cannula may not rest on the balloon and instead terminate before the balloon, so that they provide a smaller profile.

As will be appreciated by a person skilled in the art, although the embodiments described herein are described with specific features, the invention provides devices having any technically sensible combination of features described herein, for example any technically sensible combination of features from the first to fourth embodiments described herein.

A brief description of a method of use of the catheters of the invention is provided below. The method can be used for all embodiments of the device. The method may include the following steps.
1 Tracking the catheter in the deflated and needle retracted state into the desired position.
2. Confirming that catheter is in position, optionally via the use of fluoroscopy or other imaging technique.
3. Inflation of the balloon to a suitable pressure, for example a pressure of 2 atmospheres, optionally via the handle.
4. Confirming that the balloon is inflated and the guiding cannula are in position, optionally via the use of fluoroscopy or other imaging technique.
5. Manipulation of the needles from the retracted position to the extended position, optionally via the handle.
6. Infusing the substance for treatment through the needles into the target, optionally via the handle.
7. Manipulation of the needles from the extended position to the retracted position, optionally via the handle.
8. Deflation of the balloon, optionally via the handle.
9. Confirming that the balloon is deflated and the guiding cannula are in the deflated position, optionally via the use of fluoroscopy or other imaging technique.
10. Repeat steps 1-9 if more than one infusion is required.
11. After all the infusions are competed, remove the catheter from the body.

As described herein, the catheter may comprise a sheath that covers the guiding cannula, and helps to prevent the guiding cannula from damaging the vessel wall. When the catheter comprises a sheath, an exemplary method of use is as described below.
1. Tracking the catheter in the deflated and needle retracted state into the desired position.
2. Confirming that catheter is in position, optionally via the use of fluoroscopy or other imaging technique.
3. Retracting the sheath into the retracted state.
4. Inflation of the balloon to a suitable pressure, for example a pressure of 2 atmospheres, optionally via the handle.
5. Confirming that the balloon is inflated and the guiding cannula are in position, optionally via the use of fluoroscopy or other imaging technique.
6. Manipulation of the needles from the retracted position to the extended position, optionally via the handle.
7. Infusing the substance for treatment through the needles into the target, optionally via the handle.
8. Manipulation of the needles from the extended position to the retracted position, optionally via the handle.
9. Deflation of the balloon, optionally via the handle.
10. Confirming that the balloon is deflated and the guiding cannula are in the deflated position, optionally via the use of fluoroscopy or other imaging technique.
11. Extending the sheath into the extended state.
12. Repeat steps 1-11 if more than one infusion is required.
13. After all the infusions are competed, remove the catheter from the body.

In the embodiments in which a sheath is not present, there is a risk that the guiding cannula may cause damage to the vessel wall. To prevent this, a guiding catheter can be used. An exemplary method of use of the catheter in conjunction with a guiding catheter is provided below. The method can be used for all embodiments of the device, though in general will be used for embodiments that do not comprise a sheath.
1. Tracking the guiding catheter until the distal end is past the point to be treated.
2. Tracking the catheter in the deflated and needle retracted state into the desired position.
3. Confirming that catheter is in position, optionally via the use of fluoroscopy or other imaging technique.
4. Pull the guiding catheter back until the distal end of the guiding catheter is proximal to the distal end of the support structure (when present).
5. Inflation of the balloon to a suitable pressure, for example a pressure of 2 atmospheres, optionally via the handle.
6. Confirming that the balloon is inflated and the guiding cannula are in position, optionally via the use of fluoroscopy or other imaging technique.
7. Manipulation of the needles from the retracted position to the extended position, optionally via the handle.
8. Infusing the substance for treatment through the needles into the target, optionally via the handle.
9. Manipulation of the needles from the extended position to the retracted position, optionally via the handle.
10. Deflation of the balloon, optionally via the handle.
11. Confirming that the balloon is deflated and the guiding cannula are in the deflated position, optionally via the use of fluoroscopy or other imaging technique.
12. Repeat steps 1-11 if more than one infusion is required.
13. After all the infusions are competed, remove the catheter from the body.

In all embodiments of the catheter, the location of the distal end of the guiding cannula and the distal end of the support structure during use is important for catheter positioning. The position of the distal end of the guiding cannula is important because it determines where the needles will penetrate into the vessel/lumen wall. Therefore, it will be used to position the catheter at the target site. The position of the distal end of the support structure is important because it holds the components of the catheter in place, such that the parts proximal to the support structure will generally not change in dimension when the balloon inflates. Therefore, when used with a sheath or a guiding catheter, it is important to ensure that the distal end of the sheath or guiding catheter is proximal to the distal end of the support structure before balloon inflation. To aid in the visualization of the distal end of the guiding cannula during use, the entire guiding cannula can be made of a radiopaque material or radiopaque materials can be positioned at the distal end of the guiding cannula. Another possible method to allow visualization of the distal end of the guiding cannula is to have radiopaque materials within the balloon, such as on the guidewire lumen or support wire, or on any similar structure at the location of the distal end of the guiding cannula. To aid in the visualization of the distal end of the support structure during use, the entire support structure can be made of a radiopaque material or radiopaque materials can be positioned at the distal end of the support structure. To aid in the visualization of the distal end of the sheath during use, radiopaque materials can be affixed to the distal end of the sheath. The methods of allow visualization of the distal end of the guiding cannula, support structure and sheath is not limited by the method mentioned. The use of radiopaque materials described here can be applied independently, or in combination.

In the embodiments of the invention in which the distal end of the guiding cannula terminates in a curve, the curvature serves the purpose of determining the extended needles curvature and direction. In these embodiments, since the radius of curvature of the terminal end of the guiding cannula will always be smaller (i.e. a tighter curve) than that of the needle housed within, when the needle is displaced from the retracted position to the extended position, the radius of curvature of the needle will decrease as it is forced through the tighter curvature of the guiding cannula. During this process, the direction of the curvature of the needles will also take on the direction of the curvature of the guiding cannula. Having the guiding cannula set the radius and direction of curvature of the needle, allows for more precise control of the penetration depth of the needles. Another advantage of having the terminal distal end of the guiding cannula curved is that straight needles can be used, and the curved guiding cannula will force the straight needle to adopt a curved conformation upon extension out of the cannula. The radius of curvature adopted by the needles depends on the curvature present at the terminal distal end of the guiding cannula. The use the straight needles may be advantageous since it avoids deformation of the needle during storage in a straight cannula (a curved needle might be deformed and damaged by a straight cannula).

When the needles are curved outwardly when in the extended position, the force required to displace the needles from the retracted position to the extended position and vice versa can be high. This high force can be attributed to several reasons. If curved needles are used, the needles will be constantly pressed against the inner walls of the guiding cannula, and the resulting friction can require a high force to overcome. Also, when the curved needles are retracted from the extended position to the retracted position, force is required to bend the needles from a curved configuration into a straight configuration. If the terminal ends of the guiding cannula are curved, then as the needles are displaced from the retracted position to the extended position, the deformation of the needles into a smaller radius of curvature requires a significant amount of force. Due to the high forces when displacing the needles in either direction, it is important that the portion of the device which connects the distal end to the handle is made of a material that does not stretch of deform as it would affect the safety and performance of the device, Metals or alloys can provide the required properties, but can be too rigid. A way to circumvent the rigidness of metal or alloy tubes is to have the tube patterned via laser cutting to increase its flexibility. Due to the high forces required, it can be advantageous for the handle to have a mechanism which amplifies the force applied to the displacement of the needles using gears, threads, etc.

In the embodiments of the catheter in which the distal end of the guiding cannulas are free, there is the risk of the distal end of the guiding cannula causing damage to the inner wall of the lumen during the initial process of tracking the catheter to the target site. One possible solution is to have the distal end of the guiding cannulas disposed within the pleats of the folded balloon when the catheter is first inserted into the lumen and tracked to the target site. This will protect the inner wall of the lumen as it prevents the distal end of the guiding cannula from interacting directly with the inner wall of the lumen. The placement of the distal end of the guiding cannulas within the balloon pleats can be conducted either during the folding process or after. For the optimal result, the number of pleats should be equal to the number of guiding cannula as it provides the longest balloon pleat for that number of guiding cannulas. Another possible solution is to attach a soft material to the distal end of the guiding cannulas, which the soft material can cover the entire circumference of the distal end or only a select portion. The soft material can be a low durometer polymer, which soft material does not cause any obstruction to the movement of the needles. Another possible solution is to use a sheath which is movably disposed over the guiding cannula as described hereinabove. During tracking of the catheter to the target site, the sheath will be over the distal end of the guiding cannulas. Once the catheter is in position, the sheath can be retracted, exposing the distal end of the guiding cannulas to allow for unobstructed needle deployment. Yet another possible solution is to have a flexible sheath over the distal ends of the guiding cannulas. During tracking of the catheter to the target site, the flexible sheath will be over the distal end of the guiding cannulas but when the balloon is inflated, the expansion in diameter of the balloon coupled with the increase in the angle between the guiding cannula will result in the flexible sheath peeling back, thus exposing the distal end of the guiding cannulas to allow for unobstructed needle deployment. Yet another possible solution is to have a rigid sheath over the distal end of the guiding cannulas, wherein weak points can be made in the rigid sheath, wherein said weak points are located over the guiding cannulas. During tracking of the catheter to the target site, the rigid sheath will be over the distal end of the guiding cannulas but when the balloon is inflated, the expansion in diameter of the balloon will result in the rigid sheath tearing at the weak points, thus exposing the distal end of the guiding cannulas to allow for unobstructed needle deployment. As will be appreciated by a person skilled in the art, the device may comprise a combination of features intended to prevent damage to the inner wall of the vessel/lumen, and is not limited by the possible solutions mentioned. Appropriate features can be incorporated independently, or in combination.

In the embodiments of the catheter in which the distal end of the guiding cannula are free, there is a possibility that the guiding cannula does not only bend outwardly but also laterally since there is no preferential axis to bend. One possible solution is to modify the guiding cannula such that it preferentially bends in only one way. This can be achieved via various methods, for example by laser cutting one or more slits into the guiding cannula such that the guiding cannula more easily bends or adopts a certain shape. Another method is to form the guiding cannula from materials of different stiffness such that the guiding cannula will bend in a specific direction, A skilled person would understand that any method that minimizes the possibility of the guiding cannula bending laterally could be used.

In the embodiments of the catheter in which needles curved at the terminal end are used, there is a risk that prolonged storage of the needles in a straight configuration can result in the needle's radius of curvature increasing. This issue has been explored by Fischell et al in U.S. Pat. No. 8,740,849 and the proposed solution was to have the needles in the extended state during storage. When there are no obstructions at the distal opening of the guiding cannulas, the needles can be stored in the extended state during storage. However, if a sheath is present or if the guiding cannula are to be positioned within the pleats of the folded balloon, the sheath and pleats would get in the way of the needles respectively. For the sheath, one possible solution is to have the sheath in the retracted state during storage so as to not obstruct the needles. For the pleats, one possible solution would be to have the orientation of the terminal end of the needles point in an angular direction during storage to avoid the pleats while pointing in an outwardly directing during use. This can be achieved by rotating the infusion cannula in which the needles are coupled to. Said rotation conducted via the handle and the angular and outwardly positions can also be marked on the handle to ensure correct positioning. An example of how such a catheter is prepared for use would be as follows.
1. The distal ends of the guiding cannula are placed within the folded balloon pleats.
2. The orientation of the needles is set to the angular position.
3. The needles are slowly and carefully extended to ensure that the needles do not damage the balloon.
4. The catheter is packaged and stored until use.
5. Before use, the needles are retracted to the fully retracted state.
6. The orientation of the needles are set to the outwardly position.
7. The catheter can be used as per described prior.

In the embodiments of the catheter where there is a sheath, there is the possibility that air will be released from within the sheath as the sheath is displaced from the extended state to the retracted state within the lumen. The release of air can pose a risk to the patient if the lumen is a blood vessel which feeds into smaller blood vessels as the air can form bubbles and lead to air embolism. One method to significantly reduce the risk of air embolism is by allowing water to flush through the inner lumen of the sheath, preferably before the catheter is inserted into a patient's body.

In some embodiments the distal end of the guiding cannula are free and terminate along the balloon proximal cone. By having the distal ends of the guiding cannula free means that when the balloon is inflated, the inflated balloon will exert an outward force on the guiding cannula causing the free distal end to bend outwardly. Since the distal end of the guiding cannula is free, the bending of the guiding cannula will be gentle. When compared to the apparatus described by Chow et al in U.S. Pat. No. 6,692,466 and Chan et al in U.S. Pat. No. 7,273,469, this allows for smaller force to move the needles from the retracted position to the extended position and vice versa. By having the distal end of the guiding cannula terminate along the balloon proximal cone, when the balloon is fully expanded, the distal ends of the guiding cannulas will not extend in the perpendicular direction beyond the outermost diameter of the balloon. This reduces the chances of vessel injury from the contact between the guiding cannula's hollow distal end and the vessel wall. The use of a low pressure balloon also reduces the chance of vessel injury, Inflation of the balloon also positions the distal tip of the guiding cannula to be close to the inner walls of the vessel. Having the guide cannula supported by the balloon and the distal open end of the guide cannula close to the vessel inner wall allows the use of thin needles as the cannula provide radial and lateral stability for the needles as the needles are advanced through the wall of the target vessel.

A balloon may be used for the support and positioning of the guiding cannula before the deployment of the needles. Although such the use of a balloon for has been described by Mirzaee in U.S. Pat. No. 6,283,947 the rationale of the use of a balloon differ. In the prior art described by Mirzaee, the purpose of the balloon is mainly to direct the injection port angularly away from the central axis of the catheter and into the artery wall. In the present invention, the balloon serves several purposes. As mentioned prior, the expansion of the balloon is used to position and support the guiding cannula. After expansion, the balloon will fill the lumen and the balloon walls will be contact with the lumen wall. This 1) centers the catheter relative to the lumen to allow for equal penetration depth for the needles as they are extended outwardly; 2) anchors the distal end of the catheter in place within the lumen to prevent accidental repositioning; and 3) rounds out any lumens with an irregular or flattened cross sectional shape to allow for circumferential deposition of substances. This allows the present invention to be used in a variety of different physiological lumens as it is not limited to physiological lumens with a circular cross sectional area.

Needles with pre bent end portions may be used to achieve a greater penetration depth. The use of needles with pre bent end portions have been described in the art. However, known infusion catheters do not provide sufficient support for the needles, preventing the use of thin needles. The Peregrine System™ described by Fischell et al in U.S. Pat. No. 8,740,849 uses needles with pre bent end portions supported by another structure to enable the use of thin needles. The main two differences between the present invention and the Peregrine System™ is that in the present invention the guiding cannulas are positioned before the needles are deployed, and the present invention utilizes a balloon during the positioning of the cannulas and deployment of needles. These features of the invention allow it to overcome the drawbacks of the Peregrine System™ discussed above.

In embodiments and aspects of the invention described above, certain features are described in either the singular or plural. As used herein, references to the singular should be interpreted as including the plural where technically sensible, and vice versa. For example, a reference to a guiding cannula may include reference to multiple guiding cannulas where technically sensible, such as when the device comprising more than one guiding cannula.

1. A drug delivery device comprising:
an axially extending elongate member having a proximal portion, a distal portion, a first lumen and a second lumen;
an inflatable balloon coupled to the distal portion of the axially extending elongate member, the inflatable balloon having a proximal end and a distal end that are coupled together by a working portion, the balloon also having an exterior surface and an interior surface that defines a balloon chamber, where the first lumen of the axially extending elongate member is in fluid communication with the balloon chamber;
two or more guiding cannulas, each having a proximal portion and a distal portion, where the proximal portion of each guiding cannula is coupled to the axially extending elongate member,
two or more needles, each having a lumen and being housed within one of the two or more guiding cannulas, such that each needle is reversibly extendable from the distal portion of the guiding cannula that houses it, and where the second lumen of the axially extending elongate member is fluidly connectable to the lumen of each needle,
wherein, when the inflatable balloon is inflated, the distal portion of each of the two or more guiding cannulas do not extend beyond an outermost diameter of the exterior surface of the inflatable balloon.

2. The device of claim 1, wherein the inflatable balloon is coupled to the distal end of the axially extending elongate member.

3. The device of claim 2, further comprising a balloon support element within the inflatable balloon.

4. The device of claim 1, wherein the axially extending elongate member comprises a third lumen.

5. The device of claim 1, wherein the inflatable balloon is disposed over the distal portion of the axially extending elongate member.

6. The device of claim 1, wherein the distal portion of each guiding cannula is able to move freely with respect to the inflatable balloon, and/or wherein the distal portion of each guiding cannula is not affixed to the inflatable balloon.

7. The device of claim 1, wherein the guiding cannula are disposed around the circumference of the axially extending elongate member at substantially evenly spaced intervals.

8. The device of claim 1, wherein the distal portions of the guiding cannulas are configured to contact the exterior surface of the inflatable balloon and move with the exterior surface of the inflatable balloon as the inflatable balloon is inflated.

9-11. (canceled)

12. The device of claim 1, wherein each guiding cannula comprises slits cut into one side of the guiding cannula.

13. (canceled)

14. The device of claim 1, further comprising a sheath movably disposed over the axially extending elongate member.

15. The device of claim 14, wherein the sheath has an extended state and a retracted state, and wherein in the extended state the sheath covers the entirety of the two or more cannula and at least part of the inflatable balloon, and in the retracted state the sheath does not cover a part of the inflatable balloon.

16. The device of claim 1, wherein the needles are simultaneously reversibly extendable.

17-19. (canceled)

20. The device of claim 1, further comprising a support structure for positioning and securing the guiding cannulas to the axially extending elongate member.

21. The device of claim 1, wherein the distal end of the axially extending elongate member is coupled to a flexible tip.

22. The device of claim 1, wherein the device is a rapid exchange or over the wire catheter.

23. The device of claim 1, comprising at least one radiopaque element.

24. The device of claim 1, further comprising a handle disposed at the proximal end of the axially extending elongate member.

25. The device of claim 24, wherein the handle comprises a mechanism for controlling the maximum needle extension depth.

26. The device of claim 24, wherein the handle comprises a means for reversibly extending the needles from the cannulas.

27-29. (canceled)

30. The device of claim 1, wherein:
each needle comprises a curved end portion;
the distal portions of the guiding cannulas are configured to contact the exterior surface of the inflatable balloon and move with the balloon exterior as the balloon is inflated; and
the guiding cannulas are able to at least partially revert back to their original shape after the balloon is deflated.
